(19) United States
(12) Patent Application Publication
(10) Pub. No.: US 2021/0230558 A1
(43) Pub. Date: Jul. 29, 2021

(54) Dispersion of Pluripotent Stem Cells, and Pluripotent Stem Cell Product and Method for Producing Same

(71) Applicants: Sumitomo Dainippon Pharma Co., Ltd., Osaka-shi, Osaka (JP); Sumitomo Chemical Company, Limited, Tokyo (JP)

(72) Inventors: Atsushi Kuwahara, Chuo-ku, Kobe-shi, Hyogo (JP); Naoki Takamura, Chuo-ku, Kobe-shi, Hyogo (JP); Ayaka Fujiki, Chuo-ku, Kobe-shi, Hyogo (JP); Tetsuya Hayama, Chuo-ku, Kobe-shi, Hyogo (JP); Hidetaka Ohara, Suita-shi, Osaka (JP)

(73) Assignees: Sumitomo Dainippon Pharma Co., Ltd., Osaka-shi, Osaka (JP); Sumitomo Chemical Company, Limited, Tokyo (JP)

(21) Appl. No.: 17/257,719

(22) PCT Filed: Jul. 10, 2019
(86) PCT No.: PCT/JP2019/027395

§ 371 Date: Jan. 4, 2021

(30) Foreign Application Priority Data
Jul. 10, 2018 (JP) 2018-130985

(51) Int. Cl. C12N 5/074; A01N 1/02

(52) U.S. Cl. CPC C12N 5/0696; C12N 2501/999; A01N 1/0221

(57) ABSTRACT

An object of the present invention is to provide a dispersion of pluripotent stem cells and a pluripotent stem cell product having a suitable quality as a pharmaceutical or a raw material for producing a pharmaceutical which can be stored and distributed, and a method for producing the same. The method for producing a pluripotent stem cell product of the present invention comprises (1) culturing pluripotent stem cells in an undifferentiation maintenance medium; (2) suspending the pluripotent stem cells cultured in step (1) in a first medium for suspending cells containing a ROCK inhibitor; (3) replacing the medium for suspending cells of a suspension obtained in step (2) with a medium for cryopreservation to obtain a cell dispersion solution consisting of the medium for cryopreservation and the pluripotent stem cells dispersed in the medium for cryopreservation; and (4) filling the cell dispersion solution obtained in step (3) into an airtight container so as to be in an airtight state.

### TECHNICAL FIELD

The present invention relates to a dispersion of pluripotent stem cells, a pluripotent stem cell product, and a method for producing the same.

### BACKGROUND ART

Protocols for the establishment and maintenance culture of iPS cells have been published on internet, for example, by Kyoto University (Non Patent Literature 1). The iPS cells for producing master cell banks (MCBs) have been distributed, for example, by Kyoto University.

Since pluripotent stem cells such as iPS cells are expected to be used in research such as drug discovery and to be used as a pharmaceutical or a raw material for producing a pharmaceutical, their quality is very important. In order to provide a stable supply of high-quality pluripotent stem cells, it is desirable that pluripotent stem cells are produced and supplied under constant quality control.

### CITATION LIST

### Non Patent Literature

Non Patent Literature 1: Protocols Establishment and Maintenance Culture of Human iPS Cells at feeder-free (https://www.cira.kyoto-u.ac.jp/j/research/img/protocol/hipsprotocolFf_140311.pdf)

### SUMMARY OF INVENTION

### Technical Problem

An object of the present invention is to provide a dispersion of pluripotent stem cells and a pluripotent stem cell product having a suitable quality as a pharmaceutical or a raw material for producing a pharmaceutical which can be stored and distributed, and a method for producing the same.

### Solution to Problem

The present invention relates to each of the following inventions, for example.

[1] A method for producing a pluripotent stem cell product, comprising:
(1) culturing pluripotent stem cells in an undifferentiation maintenance medium;
(2) suspending the pluripotent stem cells cultured in step (1) in a first medium for suspending cells containing a ROCK inhibitor;
(3) replacing the medium for suspending cells of a suspension obtained in step (2) with a medium for cryopreservation to obtain a cell dispersion solution consisting of the medium for cryopreservation and the pluripotent stem cells dispersed in the medium for cryopreservation; and
(4) filling the cell dispersion solution obtained in step (3) into an airtight container so as to be in an airtight state.
[2] The method according to [1], wherein step (2) further includes removing the first medium for suspending cells from a suspension obtained by suspending the pluripotent stem cells in the first medium for suspending cells containing a ROCK inhibitor, and then adding a second medium for suspending cells free of a ROCK inhibitor to suspend the pluripotent stem cells.
[3] The method according to [1] or [2], wherein the ROCK inhibitor in step (2) is Y-27632.
[4] The method according to [3], wherein a concentration of Y-27632 in the first medium for suspending cells in step (2) is 100 ng/mL to 100 μg/mL.
[5] A cell dispersion consisting of a dispersion medium, and pluripotent stem cells dispersed in the medium and a ROCK inhibitor, wherein the cell dispersion is contained in an airtight container.
[6] The cell dispersion according to [5], wherein the ROCK inhibitor is Y-27632.
[7] The cell dispersion according to [6], wherein a concentration of Y-27632 in the cell dispersion is 0.01 ng/mL to 10 ng/mL.
[8] The cell dispersion according to any one of [5] to [7], wherein the cell dispersion is in a frozen state.
[9] The cell dispersion according to any one of [5] to [8], wherein airtightness of the airtight container is airtightness satisfying a criterion requiring that bacterial proliferation be not observed in a sterility test method.
[10] The cell dispersion according to [9], wherein the sterility test method is a sterility test method carried out by process simulation.
[11] The cell dispersion according to any one of [5] to [10], wherein airtightness of the airtight container is airtightness satisfying a criterion requiring that a measured value with a torque measuring device be 6.0 to 16.9 in·oz.
[12] The cell dispersion according to any one of [5] to [11], wherein a concentration of the pluripotent stem cells per unit volume of the cell dispersion is 1×10² cells/μL to 1×10⁴ cells/μL.
[13] The cell dispersion according to any one of [5] to [12], wherein the number of the pluripotent stem cells per airtight container is 2×10⁴ cells to 2×10⁷ cells, and a volume of the cell dispersion per airtight container is 200 μL to 2000 μL.
[14] The cell dispersion according to any one of [5] to [13], wherein the pluripotent stem cells are human induced pluripotent stem cells (human iPS cells) or human embryonic stem cells (human ES cells).
[15] The cell dispersion according to any one of [5] to [14], wherein when the pluripotent stem cells in the cell dispersion in a frozen state are thawed, viability of the pluripotent stem cells is at least 80%.
[16] A pluripotent stem cell product consisting of an airtight container and the cell dispersion according to any one of [5] to [15] contained in the airtight container.

### Advantageous Effects of Invention

According to the present invention, a pluripotent stem cell dispersion and a pluripotent stem cell product having a suitable quality as a pharmaceutical or a raw material for producing a pharmaceutical can be provided. Such a pluripotent stem cell dispersion has a high recovery rate and viability of pluripotent stem cells, for example, after cryopreservation and thawing.

### DESCRIPTION OF EMBODIMENTS

<Cell Dispersion>

One embodiment of the cell dispersion according to the present invention is a cell dispersion consisting of a dispersion medium, and pluripotent stem cells dispersed in the medium and a ROCK inhibitor, wherein the cell dispersion is contained in an airtight container.

### [Pluripotent Stem Cells]

The pluripotent stem cells herein are not particularly limited as long as they are stem cells having pluripotency capable of differentiating into cell lineages belonging to three germ layers (ectoderm, mesoderm, endoderm) and/or extraembryonic tissues, and also has proliferative potential.

Pluripotent stem cells can be derived from fertilized eggs, cloned embryos, germline stem cells, tissue stem cells, somatic cells, or the like. Examples of the pluripotent stem cells include embryonic stem cells (ES cells), embryonic germ cells (EG cells), and induced pluripotent stem cells (iPS cells). Multi-lineage differentiating stress enduring cells (Muse cells) obtained from mesenchymal stem cells (MSCs) and sperm stem cells (GS cells) produced from germ cells (e.g., testis) are also encompassed in pluripotent stem cells.

ES cells were first established in 1981 and have been applied to the production of knockout mice since 1989. In 1998, human ES cells were established and are being used in regenerative medicine. ES cells can be produced by culturing an inner cell mass on feeder cells. Alternatively, ES cells can also be produced by culturing an inner cell mass in a medium containing a leukemia inhibitory factor (LIF) in the case of mouse, and bFGF (fibroblast growth factor) in the case of human, instead of feeder cells. Methods for producing ES cells are described, for example, in WO 96/22362, WO 02/101057, U.S. Pat. Nos. 5,843,780, 6,200,806, and 6,280,718. ES cells can be obtained from a prescribed institution or can be purchased commercially. For example, KhES-1, KhES-2 and KhES-3, which are human ES cells, are available from the Institute for Frontier Medical Sciences, Kyoto University. EB5 and D3 cell lines, which are mouse ES cells, are available from RIKEN, National Research and Development Institute, and ATCC, respectively.

Nuclear transfer embryonic stem cells (ntES cells), one of the ES cells, can be established from cloned embryo generated by transplanting nuclei of somatic cells into enucleated eggs.

EG cells can be produced by culturing primordial germ cells in a medium containing mSCF, LIF, and bFGF (Cell, 70: 841-847, 1992).

The “induced pluripotent stem cell (iPS cell)” as used herein is a cell in which pluripotency is induced by reprogramming a somatic cell by known methods and the like. Specific examples thereof include cells that are obtained by reprogramming differentiated somatic cells such as fibroblasts or peripheral blood mononuclear cells by expression of any combination of multiple genes selected from the reprogramming gene group including Oct3/4, Sox2, Klf4, Myc (c-Myc, N-Myc, L-Myc), Glis1, Nanog, Sall4, lin28, Esrrb or the like to induce pluripotency. Preferred examples of the combination of reprogramming factors include (1) Oct3/4, Sox2, Klf4, and Myc (c-Myc or L-Myc), and (2) Oct3/4, Sox2, Klf4, Lin28, and L-Myc (Stem Cells, 2013; 31: 458-466).

iPS cells were established by Yamanaka et al. from mouse cells in 2006 (Cell, 2006, 126(4), pp. 663-676). iPS cells were also established from human fibroblasts in 2007, and have pluripotency and self-renewal ability similar to ES cells (Cell, 2007, 131(5), pp. 861-872; Science, 2007, 318 (5858), pp. 1917-1920; Nat. Biotechnol., 2008, 26(1), pp. 101-106).

In addition to the methods for producing iPS cells by directly reprogramming with gene expression, iPS cells can also be produced by the method of inducing iPS cells from somatic cells by addition of compounds or the like (Science, 2013, 341, pp. 651-654).

It is also possible to obtain established iPS cells. For example, human iPS cell lines such as 201B7 cells, 201B7-Ff cells, 253G1 cells, 253G4 cells, 1201C1 cells, 1205D1 cells, 1210B2 cells, 1231A3 cells established at Kyoto University are available from Kyoto University. As established iPS cells for clinical use, for example, Ff-I01, Ff-I14, QHJI01, and QHJI14 established at Kyoto University are available from Kyoto University.

The somatic cells used in producing iPS cells are not particularly limited, but examples thereof include fibroblasts, blood cell lineage cells (e.g., peripheral blood mononuclear cells (PBMCs), T cells), hepatocytes, pancreatic cells, intestinal epithelial cells, smooth muscle cells, and dental pulp cells derived from tissues. Examples of the source of somatic cells used in producing iPS cells include peripheral blood and umbilical cord blood collected from human blood vessels, skin tissues, and teeth.

In the production of iPS cells, when reprogramming by expression of several types of genes, the means for expressing the genes are not particularly limited. Examples of the means include infection methods with viral vector (e.g., retrovirus vector, lentivirus vector, sendai virus vector, adenovirus vector, or adeno-associated virus vector); gene transfer methods with plasmid vector (e.g., plasmid vector, or episomal vector) (e.g., calcium phosphate method, lipofection method, retronectin method, or electroporation method); gene transfer methods with RNA vector (e.g., calcium phosphate method, lipofection method, or electroporation method); and direct protein injection methods (for example, method using a needle, lipofection method, or electroporation method).

iPS cells can be produced in the presence of feeder cells or in the absence of feeder cells (feeder-free). When producing iPS cells in the presence of feeder cells, the iPS cells can be produced by a known method in the presence of a factor for maintaining undifferentiated state. The culture medium used in the production of iPS cells at feeder-free is not particularly limited, but examples thereof include a known maintenance medium for ES cells and/or iPS cells, or a medium for establishing iPS cells at feeder-free. Examples of the medium for establishing iPS cells at feeder-free include feeder-free medium such as Essential 8 medium (E8 medium), Essential 6 medium, TeSR medium, mTeSR medium, mTeSR-E8 medium, Stabilized Essential 8 medium, or StemFit medium, all of which are commercially available. iPS cells can be produced, for example, by genetically transferring four factors Oct3/4, Sox2, Klf4, and Myc into a somatic cell using sendai virus vectors at feeder-free when producing iPS cells.

The pluripotent stem cells in the present invention are preferably mammalian pluripotent stem cells, more preferably rodent (e.g., mouse or rat) or primate (e.g., human or monkey) pluripotent stem cells, and even more preferably primate pluripotent stem cells, particularly preferably human iPS cells.

[Culture Medium]

The medium used for culturing pluripotent stem cells described herein can be prepared using a medium usually used for culturing animal cells as a basal medium. Examples of the basal medium include BME medium, BGJb medium, CMRL 1066 medium, Glasgow's Minimal Essential Medium (GMEM) medium, Improved MEM Zinc Option medium, IMDM medium, Medium 199 medium, Eagle MEM medium, αMEM medium, DMEM medium, F-12 medium, DMEM/F12 medium, IMDM/F12 medium, ham medium, RPMI 1640 medium, Fischer's medium, Neurobasal medium, or a mixed medium thereof.

Herein, the medium used for culturing pluripotent stem cells is desirably a medium containing a factor for maintaining undifferentiated state (an undifferentiation maintenance medium) in order to maintain an undifferentiated state of the pluripotent stem cells. The medium can be prepared, for example, by adding a factor for maintaining undifferentiated state, a serum substitute and, as appropriate, a nutrient source to the basal medium. Specifically, the medium can be prepared by adding bFGF, KSR, non-essential amino acids (NEAA), L-glutamine, and 2-mercaptoethanol to a DMEM/F12 medium. The feeder-free medium such as Essential 8 medium (E8 medium), Essential 6 medium, TeSR medium, mTeSR medium, mTeSR-E8 medium, Stabilized Essential 8 medium, or StemFit medium as described above can also be used.

From the viewpoint of ensuring safety and ensuring suitable quality such as reducing the difference between lots, the culture medium used for culturing pluripotent stem cells is preferably a xeno-free medium, further preferably a culture medium that does not contain animal-derived components and has a chemically known composition. Thus, the medium used for culturing pluripotent stem cells is preferably a feeder-free medium or a serum-free medium.

[Dispersion Medium]

In the present description, the dispersion medium for dispersing pluripotent stem cells is not particularly limited as long as it is suitable for dispersing pluripotent stem cells, and preferably, the dispersion medium is a medium for cryopreservation which is suitable for cryopreservation.

(Medium for Cryopreservation)

The medium for cryopreservation is a dispersion medium suitable for cryopreservation of pluripotent stem cells. The medium for cryopreservation may be any medium suitable for cryopreservation of cells. Examples of the medium for cryopreservation include saline, a buffer solution such as PBS, EBSS or HBSS, a medium such as DMEM, GMEM or RPMI, serum, a serum substitute (Knock Out Serum Replacement: Invitrogen), or a mixture thereof, to each of which a cryoprotectant described below is added.

The cryoprotectant is added to maintain the function and viability of the cells as much as possible and prevent various disorders derived from freezing when performing cryopreservation of the cells. Examples of the cryoprotectant include sulfoxides such as dimethyl sulfoxide (DMSO); and linear polyols such as ethylene glycol, glycerol, propanediol, propylene glycol, butanediol, and polyethylene glycol. Preferably, oligosaccharides such as sucrose, trehalose, lactose, raffinose, and the like, are further added. Furthermore, amide compounds such as acetamide, Percoll, Ficoll 70, Ficoll 70000, polyvinylpyrrolidone and the like, may be added. When a sulfoxide is used, the sulfoxide may be added 5 to 15% (w/v), preferably 9 to 13% (w/v), more preferably around 11% (w/v) at the final concentration. When a linear polyol is used, the linear polyol may be added to the final concentration of 4 to 15% (w/v), preferably 4.5% to 8% (v/v), more preferably around 5.5% (v/v). When an oligosaccharide is used, the oligosaccharide may be added to the final concentration of 5 to 20% (w/v), preferably 8 to 12% (w/v), more preferably around 10% (w/v).

As the medium for cryopreservation, commercially available medium such as STEM-CELLBANKER (Nippon Zenyaku Kogyo Co., Ltd.), CELLBANKER 1, 1 Plus, 2, 3 (Juji Field Inc.), TC Protector (DS Pharma Biomedical Co., Ltd.), Freezing Medium for human ES/iPS Cells Co., Ltd. (ReproCELL Incorporated), CryoScarless DMSO Free (BioVerde), StemCell Keep(BioVerde), and EFS Solution (NK system) can also be used.

[ROCK Inhibitor]

To suppress cell death induced by dispersion (in particular, cell death of human pluripotent stem cells), it is preferable that the cell dispersion contain an inhibitor of Rho-associated coiled-coil kinase (ROCK) (ROCK inhibitor). It is considered that, when the cell dispersion is cryopreserved, the inclusion of a ROCK inhibitor improves the recovery rate and the viability of pluripotent stem cells upon thawing. As the ROCK inhibitor, commercially available ROCK inhibitors can be used. Examples thereof include Y-27632 (WAKO), Fasudil (HA1077), H-1152, Thiazovivin, and GSK269962, and in particular, Y-27632 is preferably used. Each ROCK inhibitor described herein includes both free form and salts or the like. For example, Y-27632 encompasses Y-27632 in free form and its hydrochloride salt such as Y-27632 dihydrochloride.

The ROCK inhibitor may be included in the cell dispersion by adding to (dissolving or dispersing in) the dispersion medium or may be included in the cell dispersion by remaining in the cell precipitate prior to dispersion in the dispersion medium. For example, when the pluripotent stem cells are dispersed in a medium for cryopreservation, it is preferable to add a ROCK inhibitor to the medium for suspending cells used for washing or the like before cryopreservation, and not to the medium for cryopreservation. In this case, when a medium for suspending cells containing a ROCK inhibitor is replaced with a medium for cryopreservation free of a ROCK inhibitor, a trace amount of the ROCK inhibitor remains, and the ROCK inhibitor is contained in the cell dispersion.

The concentration of the ROCK inhibitor in the cell dispersion can be appropriately set depending on the number of cells, the concentration of cells or the like by those skilled in the art; for example, when the ROCK inhibitor is Y-27632, the concentration may be 0.01 ng/mL to 10 ng/mL, 0.05 ng/mL to 5 ng/mL, 0.1 ng/mL to 1 ng/mL, or 0.5 ng/mL to 1 ng/mL. The concentration of the ROCK inhibitor in the cell dispersion is a concentration of the ROCK inhibitor in the supernatant obtained by centrifugation of the cell dispersion, and the concentration of the ROCK inhibitor in the supernatant can be measured by methods well known to those skilled in the art. For example, the concentration of the ROCK inhibitor in the cell dispersion can be measured by LC/MS/MS method using a calibration curve. For other ROCK inhibitor, the concentration of the ROCK inhibitor can be set from the range of concentration indicating ROCK inhibitory activity corresponding to the ROCK inhibitory activity of Y-27632 described above. The ROCK inhibitory activity can be measured by methods well known to those skilled in the art such as the ELISA method or the immunoblot method using a substrate of ROCK.

The presence of a ROCK inhibitor at the above-described concentration range in the cell dispersion can suppress the influence on differentiation induction of pluripotent stem cells upon thawing after cryopreservation and improve the recovery rate and the viability of pluripotent stem cells.

In the cell dispersion, the concentration of the pluripotent stem cells per unit volume of the cell dispersion is not particularly limited, and is preferably 1×10² cells/μL to 1×10⁴ cells/μL, and more preferably 5×10² cells/μL to 5×10³ cells/μL.

[Airtight Container]

The airtight container described herein is not particularly limited as long as it is a container for cell preservation having airtightness, i.e., a container that is free from contamination of solid or liquid foreign matters in normal handling, transport, or preserved states, and is capable of preventing loss or evaporation of the medium and cells in the container. Preferably, the airtight container is a container capable of preventing contamination of bacteria from the outside. The container capable of preventing loss or evaporation of medium and cells means a container from which the medium or the like does not leak as a liquid and from which the leakage due to evaporation is less than 1%, preferably less than 0.5%, less than 0.3%, or less than 0.1% of the weight of the medium or the like. Examples of the container include a vial and a cryotube (trade name: Nunc™ Coded Cryobank Vial Systems, manufactured by Thermo Fisher) having a cap with a gasket, and NALGENE (Nalgene) S100 cryovial or the like is also used as the container.

The airtightness of the airtight container can be confirmed by an airtightness test such as a sterility test method or a leak test. Containers for cell preservation sold as airtight containers are preferable, and those whose airtightness is guaranteed by an airtightness test are preferably used. Filling pluripotent stem cells into a container requiring airtightness confirmed by those tests is a procedure necessary to maintain suitable quality. Since airtightness also depends on the strength of the cap which tightens the container, it is necessary to tighten the cap within the range of torque values confirmed for the airtightness described above.

Sterility test methods can be performed by methods known to those skilled in the art. For example, the container of interest is filled with a medium for sterility test, subjected to static culture, and then confirmed that bacterial proliferation is not observed. Examples of the static culture include culturing at a predetermined temperature (e.g., 22.5° C., 32.5° C., or 37° C.) for several days (e.g., 5 days, 7 days, or 10 days). Regarding non-observation of bacterial proliferation, it is determined that bacterial proliferation is not observed, for example, when there is no turbidity visually or by absorbance, or the absorbance is a similar extent, compared with a control medium for sterility test.

The medium for sterility test may be any medium capable of confirming proliferation of bacteria. For example, either the liquid thioglycolic acid medium or the soybean-casein digest medium described in the Japanese Pharmacopoeia can be used (Japanese Pharmacopoeia 4.06 sterility test method).

As one embodiment of the present invention, the airtightness of the airtight container is airtightness satisfying a criterion requiring that bacterial proliferation be not observed in a process simulation sterility test method.

Process simulation in sterility test method means to simulate, among the production processes in which sterility should be ensured, a process in which bacterial contamination is likely to occur, using a medium for sterility test. Specific examples of the work in which bacterial contamination is likely to occur include, but are not limited to, thawing, medium replacement, culturing, and filling. Furthermore, it is preferable to add a load scenario that is expected in the actual production process. Examples of the load scenario include, but are not limited to, dropping a container on the floor, exchanging workers during the work, and performing the work over time. After these simulations are performed, static culture is performed, and the sterility test described above is performed. Examples of the static culture include culturing at a predetermined temperature (e.g., 22.5° C., 32.5° C., or 37° C.) for several days (e.g., 5 days, 7 days, or 10 days). It is not necessary to carry out all of the steps and load scenarios described above, and those skilled in the art can appropriately select the necessary steps and load scenarios. A sterility test by process simulation ensures not only airtightness of an airtight container, but also sterility of a dispersion of pluripotent stem cells and a pluripotent stem cell product, and a production process thereof. Thus, as one embodiment of the present invention, a dispersion of pluripotent stem cells or a pluripotent stem cell product satisfying a criterion requiring that bacterial proliferation be not observed by a sterility test in a sterility test method by process simulation is also provided.

To achieve the purpose of securing airtightness of the container, process simulation in which the container is filled with a medium for sterility test, then the container is inverted multiple times such that the medium contacts all of the inner surfaces of the container may be performed. If necessary, a load scenario such as dropping a container on the floor is added.

In another embodiment of the present invention, airtightness of an airtight container is airtightness satisfying a criterion requiring that a measured value with a torque measuring device be 6.0 to 16.9 in·oz.

Torque means moment of force around a fixed axis of rotation about which the force is applied, and represents the strength of torsion of the cap of the container. Torque is expressed as a product of force and distance. The unit of torque can be represented by a gravitational system of units (such as kgf·m), an International system of units (such as N·m), or an inch-pound system of units (such as in·lbf, in·oz, or ft·lbf). Although the unit of torque is herein represented by in·oz, those skilled in the art can easily convert each. Specifically, the units of force are converted using relationships of 1 lbf=16 oz, 1 N=0.2248 lbf, 1 kgf=9.807 N, and the units of length are converted using relationships of 1 in=2.540 cm, 1 ft=12 in.

The torque value varies depending on the material and diameter of the container; for example, when the container is a 2 mL cryotube (trade name: Nunc™ Coded Cryobank Vial Systems, manufactured by Thermo Fisher), the torque value in the range of 6.0 to 16.9 in·oz (e.g., 6.0 to 12.5 in·oz, 6.0 to 9.0 in·oz) satisfies airtightness in a sterility test method. Since the material and capacity of the container preserving the cells are similar, even when other containers are used, the range of torque values satisfying airtightness is considered to be a similar range. That is, the range of torque values may be about 75% to 150% (about 4.5 in·oz to 26 in·oz), about 80% to 150% (about 4.8 in·oz to 26 in·oz), about 80% to 125% (about 4.8 in·oz to 21 in·oz), about 90% to 125% (about 5.4 in·oz to 21 in·oz), or about 90% to 110% (about 5.4 in·oz to 19 in·oz) of the torque value of the cryotube described above, and recommended torque values are often disclosed from the manufacturer. Accordingly, those skilled in the art can deduce a certain range of torque values that satisfy airtightness from the above-described information, and can specifically confirm the torque values by a sterility test method or the like.

The capacity of the airtight container may be determined as needed, and, for example, 0.2 mL to 5 mL. The number of the pluripotent stem cells per airtight container is not particularly limited, and is preferably 1×10⁴ cells to 5×10⁷ cells, and more preferably 1×10⁵ cells to 2×10⁶ cells. The volume of the cell dispersion per airtight container is also not particularly limited, and is preferably 100 μL to 5000 μL, more preferably 200 μL to 2000 μL, and further preferably 250 μL to 1000 μL. The filling rate of the cell dispersion in the airtight container is, for example, 10% to 100%, preferably 10% to 50%.

The cell dispersion encompasses both embodiments of the cell dispersion in a liquid state (cell dispersion solution) before freezing or after freeze-thawing and the cell dispersion in a frozen state. The cell dispersion of the present invention is preferably in a frozen state. When the pluripotent stem cells in the cell dispersion in a frozen state are thawed, the viability of the pluripotent stem cells is high, and is at least 80%, at least 85%, at least 90%, at least 95%, or at least 97%. Such a high viability is suitable as a raw material for producing a pharmaceutical. Here, viability is the proportion of the number of viable cells relative to the total cells in the cryopreserved state.

<Pluripotent Stem Cell Product>

One embodiment of the pluripotent stem cell product according to the present invention consists of an airtight container and the afore-mentioned cell dispersion contained in the airtight container. The pluripotent stem cell product can be a raw material for producing a pharmaceutical and can be produced by the following methods. It is preferable that the pluripotent stem cell product be cryopreserved at, for example, −80° C. or less or −150° C. or less. The pluripotent stem cell product can be cryopreserved with liquid nitrogen (including gas phase), a deep freezer, or the like.

<Method for Producing Pluripotent Stem Cell Product>

One embodiment of the method for producing the pluripotent stem cell product according to the present invention comprises the following steps (1) to (4):

(1) culturing pluripotent stem cells in an undifferentiation maintenance medium;
(2) suspending the pluripotent stem cells cultured in step (1) in a first medium for suspending cells containing a ROCK inhibitor;
(3) replacing the medium for suspending cells of a suspension obtained in step (2) with a medium for cryopreservation to obtain a cell dispersion solution consisting of the medium for cryopreservation and the pluripotent stem cells dispersed in the medium for cryopreservation; and
(4) filling the cell dispersion solution obtained in step (3) into an airtight container so as to be in an airtight state.

All these steps are handled in an environment where microbial and particulate levels are highly restricted to prevent contamination. Preferably, the steps are carried out at a facility called a cell processing center (CPC) designed, managed and operated under certain standards. Also, the procedures in these steps are preferably sterile procedures.

[Medium for Suspending Cells]

The medium for suspending cells is used as a medium for suspending pluripotent stem cells when performing procedures such as washing or counting of the pluripotent stem cells prior to preservation of the pluripotent stem cells. The medium for suspending cells described herein is not particularly limited as long as it is suitable for suspending pluripotent stem cells. Examples of the medium for suspending cells include saline, a buffer solution such as PBS, EBSS or HBSS, a medium such as DMEM, GMEM or RPMI, serum, a serum surrogate (Knock Out Serum Replacement: Invitrogen), and a mixture thereof, and each of which may contain a cryoprotectant.

[Step (1)]

In this step, the culture includes proliferation and passage. Pluripotent stem cells for culture are typically cryopreserved when the cell lines are used. Thus, cryopreserved pluripotent stem cells are thawed prior to culture. Thawing may be performed by keeping the cells warm at 37° C. for 0.5 to 2 minutes, then the cells may be suspended in an undifferentiation maintenance medium, collected by centrifugation or the like, and seeded in a culture vessel. Pluripotent stem cells are proliferated through 1 to 3 passages from thawing to cryopreservation.

An undifferentiation maintenance medium for culturing pluripotent stem cells is a medium containing a factor for maintaining undifferentiated state as described above. Specific examples thereof include a medium containing an undifferentiation maintenance factor such as bFGF or TGFb in the basal medium described above. For example, StemFitAK03N (trade name, AJINOMOTO CO., INC.), TeSR medium (trade name, STEMCELL), Essential 8 (trade name, Thermo Fisher Scientific) or the like are preferably used.

The culture conditions may be any normal culture conditions used for culturing pluripotent stem cells, and examples thereof include a culture conditions with temperature of 37° C., CO₂ concentration of 5%, and humidity of 95%.

[Step (2)]

Step (2) is a step for washing the cells described above, wherein the pluripotent stem cells obtained in step (1) is precipitated by centrifugation or the like, the supernatant (undifferentiation maintenance medium) is removed, and then a first medium for suspending cells containing a ROCK inhibitor is added to suspend the cells. By suspending pluripotent stem cells in the first medium for suspending cells containing a ROCK inhibitor, cell death (apoptosis) of pluripotent stem cells induced by cell dispersion can be suppressed. The ROCK inhibitor is as described above, and preferably, the ROCK inhibitor is Y-27632.

The first medium for suspending cells for suspending pluripotent stem cells is a cell suspending medium suitable for suspending pluripotent stem cells and is not particularly limited as long as it contains a ROCK inhibitor. For example, the above-described medium for suspending cells includes a medium for suspending cells to which a ROCK inhibitor is added, and preferably a medium used in step (1) to which a ROCK inhibitor is added.

The concentration of the ROCK inhibitor in the first medium for suspending cells can be set as appropriate based on the desired ROCK inhibitor concentration in the finally obtained cell dispersion solution, the number of washings (i.e., dilution rate), or the like. For example, when the ROCK inhibitor is Y-27632, the concentration of the ROCK inhibitor is 50 nM to 200 μM, preferably 100 nM to 200 μM, more preferably 500 nM to 200 μM, even more preferably 1 μM to 200 μM, even more preferably 5 μM to 100 μM, even more preferably 10 μM to 30 μM. Alternatively, when the ROCK inhibitor is Y-27632, the concentration of the ROCK inhibitor is 100 ng/mL to 100 μg/mL, preferably 500 ng/mL to 10 μg/mL, further preferably 1 μg/mL to 5 μg/mL. When other ROCK inhibitors are used, the concentration of the ROCK inhibitor can be set to the range of concentrations indicating ROCK inhibitory activity corresponding to the ROCK inhibitory activity of Y-27632 at the concentration described above. When the first medium for suspending cells containing a ROCK inhibitor is replaced with a medium for cryopreservation free of a ROCK inhibitor, a trace amount of the ROCK inhibitor remains, resulting the ROCK inhibitor being contained in the cell dispersion solution.

The suspending of pluripotent stem cells in a first medium for suspending cells is carried out by recovering pluripotent stem cells present in the medium, which have been typically proliferated or passaged, by centrifugation or the like from the medium, and adding the first medium for suspending cells to the recovered cells to suspend the cells. Typically, cells are washed once, twice, three times or more with a medium for suspending cells prior to dispersing the pluripotent stem cells in a medium for cryopreservation. The washing of the cells is preferably carried out twice. Thus, the medium for cryopreservation may contain trace amounts of components of the medium for suspending cells.

The washing of cells is a procedure routinely performed by those skilled in the art. Specifically, the cells are precipitated by centrifugation, the supernatant is removed, and the cell pellet is suspended in a fresh medium (medium for suspending cells). Removal of the supernatant is carried out to the extent that the cells are not removed and the washing effect is obtained. For example, about 80%, about 85%, about 90%, about 95% of the volume of the cell suspension before centrifugation is removed. The volume of the fresh medium (medium for suspending cells) is not particularly limited, but the cell pellet is typically suspended in the fresh medium having the same volume (in the range of approximately 50% to 200%) as the volume of the cell suspension before centrifugation.

During the processes of recovery, washing and suspending in a medium of pluripotent stem cells, the pluripotent stem cells become in a dispersed state even for a short period of time, which may cause apoptosis or the like of the cells. Thus, it is preferable that the ROCK inhibitor be added to the medium for suspending cells.

The step (2) may further include replacing the medium for suspending cells in a suspension obtained by suspending the cells in the first medium for suspending cells containing a ROCK inhibitor with a second medium for suspending cells free of a ROCK inhibitor. The second medium for suspending cells may be any medium for suspending cells free of a ROCK inhibitor described above, and may be the same as or may be different from the first medium for suspending cells except for a ROCK inhibitor.

In this case, the cell suspension containing the first medium for suspending cells described above is centrifuged to remove the supernatant (the first medium for suspending cells), and then a second medium for suspending cells free of a ROCK inhibitor is added to suspend the cells. Specifically, when washing with a medium for suspending cells is performed a plurality of times, it is preferable to perform the former part of washings (at least the first time washing) with the first medium for suspending cells containing a ROCK inhibitor, and the latter part of washings (at least the last time washing) with the second medium for suspending cells free of a ROCK inhibitor. For example, when washing twice, the cells can be washed once with a first medium for suspending cells containing a ROCK inhibitor, and then washed one more time with a second medium for suspending cells free of a ROCK inhibitor. The washing with the second medium for suspending cells is a procedure to reduce the residual concentration of the ROCK inhibitor in the cell dispersion solution, which is performed in view of the possibility of the effect of the remaining ROCK inhibitor in the cell dispersion solution on the viability, differentiation or the like of the cells. However, as described above, it is preferable to suppress cell death caused by suspending during washing with a ROCK inhibitor. In other words, this procedure is a procedure which is performed in view of harmony between the two effects which are in the trade-off relationship. By this procedure, a more suitable quality of the pluripotent stem cell product can be achieved.

In step (2), it is preferable that the number of cells is counted.

[Step (3)]

The medium for cryopreservation is as described above. Replacement of the medium for suspending cells in the suspension obtained in step (2) with a medium for cryopreservation is a routine procedure for those skilled in the art, and can be performed, for example, by recovering pluripotent stem cells by centrifugation or the like, adding an appropriate amount of the medium for cryopreservation to the recovered pluripotent stem cells, and dispersing the cells in the medium to achieve a desired concentration of cells.

Depending on the number of cells, the amount of medium to be added is determined to achieve the desired concentration of cells. In the obtained cell dispersion solution, the concentration of the pluripotent stem cells per unit volume of the cell dispersion is not particularly limited, and is preferably 1×10² cells/μL to 1×10⁴ cells/μL, and more preferably 5×10² cells/μL to 5×10³ cells/μL.

As described above, a trace amount of the ROCK inhibitor remains in the cell dispersion solution. The residual concentration of the ROCK inhibitor in the cell dispersion solution depends on the concentration of the ROCK inhibitor in the first medium for suspending cells, and further, when washing with a second medium for suspending cells free of the ROCK inhibitor is performed, the number of washings and the like. Specifically, a ROCK inhibitor contained in a first medium for suspending cells is diluted, for example, 10-fold to 500-fold, 20-fold to 200-fold, or 50-fold to 100-fold in a medium for cryopreservation. When washing with a second medium for suspending cells free of a ROCK inhibitor is performed, the dilution rate of the ROCK inhibitor is a rate obtained by multiplying the above-described dilution rate by the number of washings.

Regarding the concentration of the ROCK inhibitor in the cell dispersion solution, for example, when the ROCK inhibitor is Y-27632, the residual concentration may be 0.01 ng/mL to 10 ng/mL, 0.05 ng/mL to 5 ng/mL, 0.1 ng/mL to 1 ng/mL, or 0.5 ng/mL to 1 ng/mL. The residual concentration of the ROCK inhibitor in the cell dispersion solution is the concentration of the ROCK inhibitor in the supernatant obtained by centrifugation of the cell dispersion solution, and the concentration of the ROCK inhibitor in the supernatant can be measured by methods well known to those skilled in the art. For example, the concentration of the ROCK inhibitor in the cell dispersion solution can be measured by LC/MS/MS method using a calibration curve. When the ROCK inhibitor is other than Y-27632, the concentration of the ROCK inhibitor can be set from the range of concentrations indicating ROCK inhibitory activity corresponding to the ROCK inhibitory activity described above. The ROCK inhibitory activity can be measured by methods well known to those skilled in the art, such as the ELISA method or the immunoblot method using a substrate of ROCK.

[Step (4)]

It is preferable that the cell dispersion solution obtained in step (3) is immediately filled into an airtight container. Filling so as to be in an airtight state means filling a cell dispersion solution into a container described above, and closing the cap to a torque value within a range in which the airtightness is previously confirmed.

It is preferable that the filling rate of the cell dispersion solution in the airtight container, that is, the proportion of the cell dispersion solution per volume of the airtight container, is 10 to 50%. The number of the pluripotent stem cells per airtight container varies depending on the capacity of the container or the like, and is preferably 2×10⁴ cells to 2×10⁷ cells, and more preferably 1×10⁵ cells to 2×10⁶ cells. The volume of the cell dispersion per airtight container is also not particularly limited, and is preferably 200 μL to 2000 μL, and more preferably 250 μL to 1000 μL.

[Step (5)]

The method for producing the pluripotent stem cell product may further comprise step (5), i.e., freezing a cell dispersion solution filled in an airtight container obtained in step (4), as needed. The freezing method is not particularly limited, and both the vitrification method and the slow freezing method can be used. The vitrification method is performed, for example, by immersing a tube containing cells suspended in a cryopreservation solution directly in liquid nitrogen. The slow freezing method is performed, for example, by cooling a tube containing cells suspended in a cryopreservation solution to −80° C. with a program freezer or deep freezer at −1° C./min.

It is preferable that the pluripotent stem cell product after freezing is cryopreserved at, for example, −80° C. or less, or −150° C. or less.

### EXAMPLES

Hereinafter, the present invention is described in detail with reference to Examples, but the present invention is not limited thereto.

### Example 1 Proliferation of iPS Cells

As iPS cells, Ff-I01 strains that were furnished from Kyoto University were used. The equipment used in each procedure included a safety cabinet, an incubator, a microscope, a centrifuge, or the like.

Cryopreserved Ff-I01 strains (1.0×10³ cells/μL, 200 μL/tube) were kept warm in a 37° C. water bath and thawed, then seeded in 6-well plates at 6.5×10⁴ cells/well, and cultured in an undifferentiation maintenance medium (10 μM) containing Y-27632 as a ROCK inhibitor for 24 hours. After 24 hours, the medium was replaced (medium replacement 1), and the cells were passaged, and cultured for an additional 7 days. The cultured cells were passaged, the medium was replaced (medium replacement 2), and after 7 days, the cells were recovered, counted, and the medium was replaced with a medium for cryopreservation (STEM-CELLBANKER). The concentration of the obtained cell dispersion solution was 1.0×10³ cells/μL.

### Example 2 Filling and Cryopreservation of iPS Cells in Airtight Container

The cell dispersion solution obtained in Example 1 was filled into 2 mL cryovials in a safety cabinet to become 250 μL/tube, 500 μL/tube, and 1000 μL/tube. After filling, the cells were cryopreserved with a program freezer.

### Example 3 Cell Viability after Thawing

The cryopreserved iPS cells in Example 2 were thawed as in Example 1. The number of viable cells after thawing was counted, taken as the number of recovered cells, and the recovery rate was calculated, and further the viability in the recovered cells was calculated. Thawed cells were seeded in 6-well plates at 6.5×10⁴ cells/well, cultured for 4 days in an undifferentiation maintenance medium (containing ROCK inhibitor Y-27632 at 10 μM for the first 24 h only), the number of recovered cells was counted, and the viability was calculated. The results are shown in Tables 1 and 2.

TABLE 1
Number of recovered cells, recovery rate and viability after thawing

| Amount of | Number of | Number of | Recovery | Viability |
|---|---|---|---|---|
| frozen liquid | frozen cells | recovered cells | rate (%) | (%) |
| (μL/tube) | (cells/tube) | (mean ± SD) | (mean ± SD) | (mean ± SD) |
| 250 | 2.5 × 10⁵ | 1.36 × 10⁵ ± | 54.3 ± 3.3 | 97.0 ± 1.73 |
| | | 8.30 × 10³ | | |
| 500 | 5.0 × 10⁵ | 2.93 × 10⁵ ± | 58.6 ± 3.1 | 98.5 ± 0.87 |
| | | 1.53 × 10⁴ | | |
| 1000 | 1.0 × 10⁶ | 6.50 × 10⁵ ± | 65.0 ± 1.9 | 98.8 ± 0.29 |
| | | 1.93 × 10⁴ | | |

TABLE 2
Number of recovered cells and viability after 4-day culture

| Amount of | Number of frozen | Number of | Recovery |
|---|---|---|---|
| frozen liquid | cells | recovered cells | rate (%) |
| (μL/tube) | (cells/tube) | (mean ± SD) | (mean ± SD) |
| 250 | 2.5 × 10⁵ | 7.63 × 10⁵ ± 1.94 × 10⁴ | 95.2 ± 1.15 |
| 500 | 5.0 × 10⁵ | 7.61 × 10⁵ ± 2.63 × 10⁴ | 96.0 ± 0.50 |
| 1000 | 1.0 × 10⁶ | 6.65 × 10⁵ ± 3.73 × 10⁴ | 95.0 ± 1.00 |

From Tables 1 and 2, 95% or more viability after thawing and viability after 4-day culture were observed at any amount of frozen liquid.

### Example 4 Airtightness Test 1

Instead of the cell dispersion solution, a medium for sterility test was used, and the relatively risky work among the work from thawing to filling, namely thawing, passage, medium replacement 2 and filling, were simulated, and the sterility of the production process was checked. It should be noted that the following load scenarios, which are expected to a certain degree, were added to each step. This test was performed twice.

### [Load Scenarios]

Thawing: The tube was dropped on the floor, then wiped with actril, and then returned to a safety cabinet.
Passage: The tube was dropped on the floor, then wiped with actril, and then returned to a safety cabinet. The assistant put the hand deeply into the interior of the safety cabinet.
Medium replacement: A worker newly entered the cell preparation room. The workers were exchanged.
Filling: The work was performed for 60 minutes or more.

As a result, bacterial proliferation was not observed in all of the first and second specimens. The highly airtightness of the vials was proved.

### Example 5 Measurement of Concentration of ROCK Inhibitor

In Sumitomo Dainippon Pharma Co., Ltd., established iPS cell lines were produced by the conventional methods described in the present description. The iPS cells were washed once with a culture medium (AK03N: AJINOMOTO CO., INC.) containing Y-27632 (10 μM), and washed one more time with a culture medium (AK03N:AJINOMOTO CO., INC.) containing no Y-27632. After washing, iPS cells were cryopreserved as a cell bank. Y-27632 contained in the supernatant of frozen iPS cells was quantified using each one of the two lots of the cell bank produced as described above as specimen. The samples were centrifuged and then the supernatant was sampled. The samples were measured by LC/MS/MS method using 0.0400 to 20.0 ng/mL Y-27632 solutions as calibration curve samples. As a result, it was found that specimen 1 contained 0.819 ng/mL Y-27632 and specimen 2 contained 0.864 ng/mL Y-27632.

1. A method for producing a pluripotent stem cell product, comprising:
(1) culturing pluripotent stem cells in an undifferentiation maintenance medium;
(2) suspending the pluripotent stem cells cultured in step (1) in a first medium for suspending cells containing a ROCK inhibitor;
(3) replacing the medium for suspending cells of a suspension obtained in step (2) with a medium for cryopreservation to obtain a cell dispersion solution consisting of the medium for cryopreservation and the pluripotent stem cells dispersed in the medium for cryopreservation; and
(4) filling the cell dispersion solution obtained in step (3) into an airtight container so as to be in an airtight state.

2. The method according to claim 1, wherein step (2) further includes removing the first medium for suspending cells from a suspension obtained by suspending the pluripotent stem cells in the first medium for suspending cells containing a ROCK inhibitor, and then adding a second medium for suspending cells free of a ROCK inhibitor to suspend the pluripotent stem cells.

3. The method according to claim 1, wherein the ROCK inhibitor in step (2) is Y-27632.

4. The method according to claim 3, wherein a concentration of Y-27632 in the first medium for suspending cells in step (2) is 100 ng/mL to 100 μg/mL.

5. A cell dispersion consisting of a dispersion medium, and pluripotent stem cells dispersed in the medium and a ROCK inhibitor, wherein the cell dispersion is contained in an airtight container.

6. The cell dispersion according to claim 5, wherein the ROCK inhibitor is Y-27632.

7. The cell dispersion according to claim 6, wherein a concentration of Y-27632 in the cell dispersion is 0.01 ng/mL to 10 ng/mL.

8. The cell dispersion according to claim 5, wherein the cell dispersion is in a frozen state.

9. The cell dispersion according to claim 5, wherein airtightness of the airtight container is airtightness satisfying a criterion requiring that bacterial proliferation be not observed in a sterility test method.

10. The cell dispersion according to claim 9, wherein the sterility test method is a sterility test method carried out by process simulation.

11. The cell dispersion according to claim 5, wherein airtightness of the airtight container is airtightness satisfying a criterion requiring that a measured value with a torque measuring device be 6.0 to 16.9 in·oz.

12. The cell dispersion according to claim 5, wherein a concentration of the pluripotent stem cells per unit volume of the cell dispersion is 1×10² cells/μL to 1×10⁴ cells/μL.

13. The cell dispersion according to claim 5, wherein the number of the pluripotent stem cells per airtight container is 2×10⁴ cells to 2×10⁷ cells, and a volume of the cell dispersion per airtight container is 200 μL to 2000 μL.

14. The cell dispersion according to claim 5, wherein the pluripotent stem cells are human induced pluripotent stem cells (human iPS cells) or human embryonic stem cells (human ES cells).

15. The cell dispersion according to claim 5, wherein when the pluripotent stem cells in the cell dispersion in a frozen state are thawed, viability of the pluripotent stem cells is at least 80%.

16. A pluripotent stem cell product consisting of an airtight container and the cell dispersion according to claim 5 contained in the airtight container.
